(19) United States
(12) Patent Application Publication
(10) Pub. No.: US 2023/0263710 A1
(43) Pub. Date: Aug. 24, 2023

(54) ORAL CARE AGENT

(71) Applicants: OSAKA UNIVERSITY, Suita-shi, Osaka (JP); ACENET INC., Tokyo (JP)

(72) Inventors: Takayoshi SAKAI, Osaka (JP); Tsuyoshi INOUE, Osaka (JP); Kiyoto TAKAMORI, Tokyo (JP); Takekatsu SHIBATA, Tokyo (JP)

(21) Appl. No.: 18/005,552

(22) PCT Filed: Jul. 15, 2021
(86) PCT No.: PCT/JP2021/026617

§ 371 Date: Jan. 13, 2023

(30) Foreign Application Priority Data
Jul. 16, 2020 (JP) 2020-122198

(51) Int. Cl. A61K 8/20; A61K 8/41; A61Q 11/00

(52) U.S. Cl. CPC A61K 8/20; A61K 8/416; A61Q 11/00

(57) ABSTRACT

The present invention provides an oral care agent suitable for removing oral deposits. An oral care agent of the present invention is a semi-solid oral care agent that is an agent for removing oral deposits.

### TECHNICAL FIELD

The present invention relates to an oral care agent.

### BACKGROUND ART

Examples of oral deposits include oral mucosae such as sputum, crust, and exfoliated epithelium. These are, for example, found in the oral cavities of elderly people requiring nursing care, and there is a possibility that the oral cavity can become a place for bacterial contamination, which can lead to pneumonia (Non-Patent Literature 1).

### CITATION LIST

### Non-Patent Literature

Tadashi Ogasawara, Yuka Kawase, Kazushige Isono, Yoshiyuki Okada, Hiroyuki Haishima, Fa-Chih Shen, Mami Endo, Takanaga Ochiai, Hiromasa Hasegawa, Yasuaki Kakinoki, “Formative Factors of Membranous Substances on Dorsum of Tongue, Teeth, Buccal Mucosa in Elderly Persons Requiring Nursing Care”, RONEN SHIKA IGAKU, 2014, Volume 29, Issue 1, Pages 11 to 20

### SUMMARY OF INVENTION

### Technical Problem

However, oral deposits are difficult to be remove because they often adhere to the oral mucosa, tongue, gingivae, teeth, and the like.

With the foregoing in mind, it is an objective of the present invention to provide an oral care agent suitable for removing oral deposits.

### Solution to Problem

In order to achieve the above objective, the present invention provides a first oral care agent, being a semi-solid oral care agent, that is an agent for removing oral deposits.

The present invention also provides a second oral care agent, being an oral care agent, that includes: a radical generator, wherein the oral care agent is an agent for removing oral deposits. In the following description, the first oral care agent of the present invention and the second oral care agent of the present invention may be collectively referred to as the “oral care agent of the present invention”. In the following description, the term “oral care agent of the present invention” includes both the first oral care agent of the present invention and the second oral care agent of the present invention, and may be either the first oral care agent of the present invention or the second oral care agent of the present invention, unless otherwise specified.

### Advantageous Effects of Invention

According to the present invention, it is possible to provide an oral care agent suitable for removing oral deposits.

### BRIEF DESCRIPTION OF DRAWINGS

FIG. 1 (A) to (D) of FIG. 1 are photographs showing a process of applying an oral care agent to oral deposits mainly composed of sputum and exfoliated epithelium, clot, bacteria, virus, dental plaque, and biofilm adhered to and hardened on the palatal mucosa to soften and removing them with a sponge brush.

FIG. 2 is a photograph showing a process of applying an oral care agent to oral deposits mainly composed of sputum and exfoliated epithelium, clot, bacteria, virus, dental plaque, and biofilm adhered to and hardened on the surfaces of teeth and gingivae like calculus to soften and gradually removing by suction while crushing them with a toothbrush.

FIG. 3 (A) to (D) of FIG. 3 are photographs showing the state after about 5 to 10 minutes from the application of the oral care agent. While oral deposits adhered to and hardened on the surfaces of teeth and gingivae like calculus cause not only caries and periodontal disease but also pneumonia and asphyxiation, the effect of the oral care agent allows the oral deposits to be gradually softened from the peripheral portion and safely removed as a lump without bleeding or aspiration by using a sponge brush as shown in (A) to (D) of FIG. 3.

FIG. 4 (A) of FIG. 4 is a photograph showing the exfoliated epithelial piece after being immersed in water for 2 hours. (B) of FIG. 4 is a photograph showing the exfoliated epithelial piece after being in an agent (200 ppm MA-Tγ) including a radical generator (sodium chlorite) and a radical generating catalyst (benzalkonium chloride) for 2 hours.

FIG. 5 (A) of FIG. 5 is a photograph showing the exfoliated epithelial piece after being immersed in water for 24 hours. (B) of FIG. 5 is a photograph showing the exfoliated epithelial piece after being immersed in an agent (200 ppm MA-Tγ) including a radical generator (sodium chlorite) and a radical generating catalyst (benzalkonium chloride) for 24 hours.

### DESCRIPTION OF EMBODIMENTS

Hereinafter, the present invention will be described in more detail with reference to examples. The present invention, however, is not limited by the following descriptions.

In the present invention, when a compound (e.g., ammonium or the like to be described below) has an isomer such as a tautomer or a stereoisomer (e.g., a geometric isomer, a conformer, and an optical isomer), any isomer can be used unless otherwise specified. In addition, in the present invention, when a substance (for example, an oxo acid, an oxo acid ion, a radical generating catalyst, and the like to be described below) can form a salt, the salt can also be used unless otherwise specified. The salt may be an acid addition salt or a base addition salt. Furthermore, the acid forming the acid addition salt may be an inorganic acid or an organic acid, and the base forming the base addition salt may be an inorganic base or an organic base. Examples of the inorganic acid include, but are not limited to, sulfuric acid, phosphoric acid, hydrofluoric acid, hydrochloric acid, hydrobromic acid, hydriodic acid, hypofluorous acid, hypochlorous acid, hypobromous acid, hypoiodous acid, fluorine acid, chlorous acid, bromous acid, iodous acid, fluoric acid, chloric acid, bromic acid, iodic acid, perfluoric acid, perchloric acid, perbromic acid, and periodic acid. Examples of the organic acid include, but are not limited to, p-toluenesulfonic acid, methanesulfonic acid, oxalic acid, p-bromobenzenesulfonic acid, carbonic acid, succinic acid, citric acid, benzoic acid, and acetic acid. Examples of the inorganic base include, but are not limited to, ammonium hydroxide, alkali metal hydroxides, alkaline earth metal hydroxides, carbonates, and hydrogencarbonates, and more specifically, sodium hydroxide, potassium hydroxide, potassium carbonate, sodium carbonate, sodium hydrogencarbonate, potassium hydrogencarbonate, calcium hydroxide, and calcium carbonate. Examples of the organic base include, but are not limited to, ethanolamine, triethylamine, and tris(hydroxymethyl)aminomethane. The method for producing these salts is not particularly limited, and for example, the compound can be produced by adding the acid or the base as described above to the compound by a suitable known method.

Furthermore, in the present invention, a chain substituent (e.g., a hydrocarbon group such as an alkyl group, an unsaturated aliphatic hydrocarbon group, and the like) may be either linear or branched, unless otherwise specified, and the number of carbon thereof may be, but not limited to, for example 1 to 40, 1 to 32, 1 to 24, 1 to 18, 1 to 12, 1 to 6, or 1 to 2 (2 or more in the case of an unsaturated hydrocarbon group). In the present invention, the number of ring members (the number of atoms constituting the ring) of a cyclic group (e.g., an aryl group, a heteroaryl group, and the like) is not particularly limited, and may be, for example, 5 to 32, 5 to 24, 6 to 18, 6 to 12, or 6 to 10. When a substituent or the like has an isomer, the isomer may be any isomer unless otherwise specified, and for example, when simply referred to as a “naphthyl group”, the isomer may be a 1-naphthyl group or a 2-naphthyl group.

[1. Oral Care Agent]

As described above, the first oral care agent of the present invention is a semi-solid oral care agent which is an agent for removing oral deposits. As described above, the second oral care agent of the present invention is an oral care agent including: a radical generator, wherein the oral care agent is an agent for removing oral deposits.

In the oral care agent of the present invention, the oral deposits are not particularly limited. The oral deposit may be, for example, a substance adhered to the oral mucosa, tongue, gingivae, or teeth. In the present invention, the “oral mucosa” includes, but is not limited to, for example, the entire mucosa in the oral cavity, and examples thereof include mucosa of lingual and labial sides of the gingiva, maxilla, mandible, and the like. Further, the deposit on the tongue includes, but is not limited to, for example, deposits on the entire tongue, and examples thereof include deposits on the front surface of the tongue and deposits on the back surface of the tongue. The oral deposits may be, but not limited to, at least one substance selected from the group consisting of sputum, crust, exfoliated epithelium, clot, bacteria, virus, dental plaque, and biofilm. The biofilm may be, but not limited to, a biofilm formed from one type or two or more types of oral deposits such as sputum, crust, exfoliated epithelium, clot, bacteria, virus, dental plaque, and the like.

In the oral care agent of the present invention, the term “semi-solid” means that the oral care agent has an intermediate property between liquid and solid and can maintain a constant shape like a solid, and at the same time, is viscous and freely deformable like a liquid. In the oral care agent of the present invention, the term “semi-solid” may mean, for example, a state of having thixotropic and rheological properties, and of being changeable to a high viscosity state (gel) and a low viscosity state (sol). More specifically, the “semi-solid” in the oral care agent of the present invention means preferably, for example, a state of being capable of applying to the oral cavity and remaining on the applied surface without flowing down therefrom as it is. In the present invention, the “semi-solid” may be, for example, a gel form or a state similar to an ointment, a toothpaste, or the like. The viscous property of the “semi-solid” in the oral care agent of the present invention can be expressed, for example, in viscosity, which is the degree of viscous property of the fluid. The first oral care agent of the present invention is semi-solid as described above, and the viscosity thereof is not particularly limited. The second oral care agent of the present invention may be, for example, liquid or semi-solid.

The use of the oral care agent of the present invention is not particularly limited except that it is used for removing oral deposits, and may be used, for example, for prevention or treatment of an oral disease. Examples of the oral disease include, but are not limited to, caries, periodontal disease, stomatitis, and oral candidiasis.

Also, the method for using the oral care agent of the present invention is not limited to application, and is optional. The oral care agent of the present invention may be used, for example, as a dentifrice. For example, when the oral care agent of the present invention has an antibacterial activity against the causative bacteria of caries and periodontal disease, it can be used as a dentifrice also for the purpose of prevention or treatment of caries and periodontal disease. A person who can gargle by himself/herself may gargle by himself/herself using the oral care agent of the present invention having a low viscosity. For example, when the oral care agent of the present invention has antibacterial activity against bacteria, antiviral activity against viruses, and the like, the oral care agent of the present invention can be used for gargling to prevent bacterial infection, viral infection, and the like. Further, for example, a patient who cannot gargle, a patient who cannot swallow, or the like may be treated (application, cleaning, toothbrushing, or the like) with the oral care agent of the present invention having a high viscosity by a dentist, a dental hygienist, a doctor, a nurse, a linguistic auditor, or the like in order to prevent aspiration. This makes it possible, for example, to prevent pneumonia and asphyxiation.

The oral care agent of the present invention may be, for example, a semi-solid base to which other components are added. The base is not particularly limited, and may be, for example, chlorhexidine gluconate, and the like, and one type of the base maybe used alone, or two or more of types of the base may be used in combination. The base may be, for example, a solvent to which a thickener is added. The solvent may be, for example, water, an organic solvent, or a mixture of water and an organic solvent. The organic solvent may be, for example, ethanol. The thickener is not particularly limited, and may be, for example, a natural thickener, a semi-synthetic thickener, or a synthetic thickener, and one type of the thickener maybe used alone, or two or more types of the thickener may be used in combination. Examples of the natural thickener include a plant thickener, a microbial thickener, and an animal thickener. Examples of the plant thickener include gum arabic, tragacanth, and carrageenan. The microbial thickener may be, for example, xanthan gingiva. Examples of the animal thickener include gelatin, chitosan, and sodium chondroitin sulfate. Examples of the semi-synthetic thickener include a cellulose thickener, a starch thickener, and an alginic acid thickener. Examples of the cellulosic thickener include methylcellulose, carboxymethylcellulose, sodium nitrocellulose, ethylcellulose, hydroxyethylcellulose, and hydroxypropylcellulose. Examples of the starch thickener include rice starch, wheat starch, and dextrin. Examples of the alginate thickener include sodium alginate and propylene glycol alginate. Examples of the synthetic thickener include vinyl thickeners, acrylic thickeners, silicone thickeners, and other thickeners. Examples of the vinyl thickener include polyvinyl alcohol, polyvinyl pyrrolidone, and polyvinyl acetate. Examples of the acrylic thickener include sodium polyacrylate, polyacrylic resin alkanolamine solution, polyethyl methacrylate, and carboxyvinyl polymer. Examples of the silicone thickener include polymethylsiloxane and methylphenylpolysiloxane. Examples of other thickeners include polyethylene glycol, polyoxyethylene-polyoxypropylene copolymer, propylene glycol fatty acid ester, and glycerin fatty acid ester. Examples of the thickener include xylitol, sorbitol, carboxymethylcellulose (CMC), and hydroxyethylcellulose (HEC). The content of the thickener is not particularly limited, and may be, for example, 0.1% by mass or more, 1% by mass or more, 2% by mass or more, 3% by mass or more, or 5% by mass or more, and may be, for example, 10% by mass or less, 8% by mass or less, 5% by mass or less, 3% by mass or less, or 2% by mass or less with respect to the total mass of the solvent. Components other than the base are not particularly limited, and may be, for example, an active ingredient as an agent or a further component, and one type of the components maybe used alone or two or more types of the components may be used in combination. Other components may include, for example, an antimicrobial component, an antiviral component, a radical generator, a radical generating catalyst, and the like, which will be described below.

The oral care agent of the present invention may or may not include, for example, at least one of an antimicrobial component and an antiviral component. The content of at least one of the antibacterial component and the antiviral component is not particularly limited, and may be, for example, 0.0001% by mass or more, 0.001% by mass or more, 0.01% by mass or more, 0.1% by mass or more, or 1% by mass or more, and may be, for example, 10% by mass or less, 8% by mass or less, 5% by mass or less, 3% by mass or less, or 2% by mass or less with respect to the total mass of the oral care agent of the present invention. One type of the antimicrobial components and the antiviral components may be used alone or two or more types of them may be used in combination. A component that acts as both an antimicrobial component and an antiviral component may be used, for example. The antimicrobial component and the antiviral component may be, for example, radical generators to be described below, and examples thereof include isopropylmethylphenol (IPMP, sold under the trade name “Biozole” by Osaka Kasei Co., Ltd.), cetylpyridinium chloride, and ethanol.

As described above, other than being used for removing oral deposits, the use of the oral care agent of the present invention is not particularly limited. The oral care agent of the present invention may be used, for example, for prevention or treatment of infectious disease, suppression of seriousness, or suppression of infection to other people. The infectious disease is not particularly limited, and may be, for example, a respiratory tract infection. The respiratory tract infection is not particularly limited, and may be, for example, a respiratory tract infection caused by various microorganisms, viruses, and the like (for example, coronavirus or influenza virus) to be described below. For example, as described above, when the oral care agent of the present invention has antibacterial activity against bacteria, antiviral activity against viruses, and the like, the oral care agent of the present invention can be used for gargling to prevent bacterial infection, viral infection, and the like. The oral care agent of the present invention may have an antibacterial activity against bacteria, for example, by including an antibacterial component. For example, the oral care agent of the present invention may have an antiviral ability against a virus by including an antiviral component.

In the present invention, the term “infection” or “infectious disease” refers to a nature of a microorganism or a virus to stably grow in a host. The microorganism means, for example, a prokaryotic bacterium, a eukaryotic fungus, or the like. In addition, in the present invention, the term “pathogen” means, for example, a pathogen having DNA or RNA and causing a disease of an organism. Examples of the pathogen include coronaviruses (including, for example, SARS-CoV, the causative virus of SARS, SARS-CoV-2, the causative virus of COVID-19, and MERS-CoV, the causative virus of MERS), influenza virus, norovirus, hepatitis B virus, hepatitis C virus, mycoplasma, Staphylococcus aueus, Streptococcus pneumoniae, Haemophilus influenzae, ESBL-producing bacteria, Neisseria gonorrhoeae, Mycobacterium tuberculosis, hemolytic streptococci, Clostridium difficile, enterobacteria, enterococci, Acinetobacter, Pseudomonas aeruginosa, Staphylococcus aureus, Campylobacter, Candida, nontyphoidal salmonella, Salmonella typhi, Shigella, Group A Streptococcus, Group B Streptococcus, nontuberculous mycobacteria, spirochetes, and fungi.

In the present invention, the term “antimicrobial” is not limited to suppression of the growth of microorganisms, and should be interpreted in the broadest sense including sterilization, decolonization, disinfection, total sterilization, bacteriostatic, and the like, and is not limited in any sense. The term “sterilization” means, for example, killing of microorganisms. The term “decolonization” means, for example, the removal and reduction of microorganisms. The term “disinfection” means, for example, killing or reducing microorganisms that are pathogenic to an animal such as a human or plant to detoxify them. The term “total sterilization” means, for example, killing of all microorganisms. The term “bacteriostatic” means, for example, inhibiting or preventing the growth of microorganisms.

In the present invention, the term “antivirus” should be interpreted in the broadest sense including decrease of infectivity of a virus, prevention of infection of a virus, inactivation of a virus, inhibition of growth of a virus, and the like, and is not limited in any sense.

The first oral care agent of the present invention may or may not include, for example, a radical generator. The second oral care agent of the present invention includes a radical generator as described above. In the oral care agent of the present invention, the radical generator may also serve, for example, at least one component of the antimicrobial component or the antiviral component. The content of the radical generator is not particularly limited, and may be, for example, 0.0001% by mass or more, 0.001% by mass or more, 0.01% by mass or more, or 1% by mass or more, and may be, for example, 1% by mass or less, 0.5% by mass or less, 0.3% by mass or less, or 0.2% by mass or less with respect to the total mass of the oral care agent of the present invention. One type of the radical generators may be used alone or two or more types of them may be used in combination.

The radical generator may include, for example, at least one material selected from the group consisting of oxo acid, oxo acid ion, and oxo acid salt.

The oxo acid may be, for example, at least one material selected from the group consisting of boric acid, carbonic acid, orthocarbonic acid, carboxylic acid, silicic acid, nitrous acid, nitric acid, phospholipid acid, phosphoric acid, arsenic acid, sulfurous acid, sulfuric acid, sulfonic acid, sulfinic acid, chromic acid, nichromic acid, permanganic acid, and halogenoxo acid.

The oxo acid may be, for example, a halogen oxo acid. The halogen oxo acid may be, for example, at least one material selected from the group consisting of chlorine oxo acid, bromine oxo acid, and iodine oxo acid. The halogen oxo acid may be, for example, chlorine oxo acid.

The halogen oxo acid may be, for example, at least one material selected from the group consisting of hypochlorous acid, chlorous acid, chloric acid, perchloric acid, hypobromous acid, bromous acid, bromic acid, perbromic acid, hypoiodous acid, iodous acid, iodic acid, and periodic acid. The halogen oxo acid may be, for example, at least one material selected from the group consisting of hypohalous acid, halous acid, halogen acid, and perhalogen acid. The halogen oxo acid may be, for example, halous acid. The halogen oxo acid may be, for example, chlorite.

In the oral care agent of the present invention, when the oxo acid is in the form of an oxo acid salt, the oxo acid salt is not particularly limited, and may be an inorganic salt or an organic salt. The inorganic salt and the organic salt are not particularly limited, and specific examples thereof are as described above.

The oral care agent of the present invention may further include, for example, a radical generating catalyst, and the radical generating catalyst may be a substance that catalyzes radical generation from the radical generator included in the oral care agent. However, the oral care agent of the present invention may or may not include the radical generating catalyst. In the following description, the radical generating catalyst may be referred to as the “radical generating catalyst of the present invention”.

The radical generating catalyst of the present invention may be, for example, an organic compound or an inorganic substance. The organic substance may be, for example, at least one material selected from the group consisting of ammonium, amino acids, proteins, peptides, phospholipids, and salts thereof. The inorganic substance may include one or both of metal ions and nonmetal ions. The metal ion may include one or both of typical metal ions and transition metal ions. The inorganic substance may be, for example, at least one selected from the group consisting of alkali earth metal ions, rare earth ions, Mg²⁺, Sc³⁺, Li⁺, Fe²⁺, Fe³⁺, Al³⁺, silicate ions, and borate ions. Examples of the alkali earth metal ion include ions of calcium, strontium, barium, and radium. More specifically, examples of the alkali earth metal ion include Ca²⁺, Sr²⁺, Ba²⁺, and Ra²⁺. Furthermore, the “rare earth metal” is a generic name of a set of seventeen elements, specifically, two elements such as scandium₂₁Sc and yttrium₃₉Y and fifteen elements (lanthanoids) from lanthanum₅₇La to lutetium₇₁Lu. Examples of the rare earth ion include corresponding trivalent cations of the seventeen elements.

The radical generating catalyst may be, for example, at least one selected from the group consisting of CaCl₂, MgCl₂, FeCl₂, FeC₃, AlC₃, AlMeCl₂, AlMe₂Cl, BF₃, BPh₃, BMe₃, TiCl₄, SiF₄, and SiCl₄. It is to be noted that the “Ph” represents a phenyl group, and the “Me” represents a methyl group.

The radical generating catalyst may be, for example, a Lewis acid having a Lewis acidity of no lower than 0.4 eV. The radical generating catalyst may be, for example, Brønstead acid having an acid dissociation constant pKa of 5 or more as Brønstead acid. The upper limit of pKa is not particularly limited, and is, for example, 50 or less.

In the radical generating catalyst of the present invention, the radical generating catalyst can be selected appropriately depending on the intended use thereof, with consideration given to the reactivity, acidity, safety, and the like.

In the present invention, although the reason why the ammonium, amino acids, peptides, and phospholipids function as radical generating catalysts is not clear, it is presumably because the ammonium, amino acids, peptides, and phospholipids each have a function as a Lewis acid. In the present invention, the “Lewis acid” refers to, for example, a substance that serves as a Lewis acid with respect to the radical source.

The Lewis acidity of the radical generating catalyst of the present invention is, for example, 0.4 eV or more, 0.5 eV or more, or 0.6 eV or more. The upper limit of the Lewis acidity is not particularly limited, and is, for example, 20 eV or less. In the present invention, a criterion for determining that the Lewis acidity is equal to, greater than, or less than the above-described numerical value is, for example, whether the measured value by any one of the “Lewis acidity measuring method (1)” and the “Lewis acidity measuring method (2)” described below is equal to, greater than, or less than the above-described numerical value.

The Lewis acidity can be measured, for example, by the method described in Ohkubo, K.; Fukuzumi, S. Chem. Eur. J., 2000, 6, 4532, J. Am. Chem. Soc. 2002, 124, 10270-10271 or the method described in J. Org. Chem. 2003, 68, 4720-4726. Specifically, the Lewis acidity can be measured by the following “Lewis acidity measuring method (1)”.

(Lewis Acidity Measuring Method (1))

As to acetonitrile (MeCN) that contains cobalt tetraphenylporphyrin, saturated O₂, and an object whose Lewis acidity is to be measured (e.g., a cation of a metal or the like, represented by Mⁿ⁺ in the following chemical reaction formula (1a)) in the following chemical reaction formula (1a), the change of the ultraviolet-visible absorption spectrum is measured at room temperature. On the basis of the obtained reaction rate constant (k_{cat}), the ΔE value (eV), which is an indicator of the Lewis acidity, can be calculated. The higher the k_{cat}, the stronger the Lewis acidity. Furthermore, the Lewis acidity of an organic compound can be estimated from the energy level of the lowest unoccupied molecular orbital (LUMO) calculated by the quantum chemical calculation. The higher the value at the positive side, the stronger the Lewis acidity.

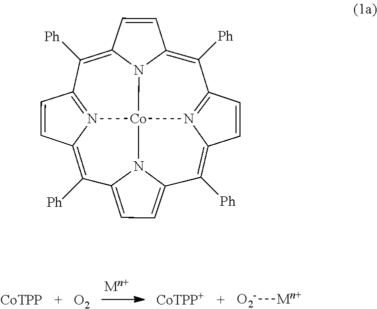

Examples of the reaction rate constant of reaction between CoTPP and oxygen in the presence of a Lewis acid, which is an indicator of the Lewis acidity measured (calculated) by the above-described measurement method, are shown below. In the following table, the numerical value expressed in the unit “k_{cat}, M⁻² s⁻¹” is the reaction rate constant of reaction between CoTPP and oxygen in the presence of a Lewis acid. The numerical value expressed in the unit “LUMO, eV” is the energy level of LUMO.

TABLE tpp

| | LUMO, eV | k_{cat}, M⁻² s⁻¹ |
|---|---|---|
| benzethonium chloride | −4.12 | 0.24 |
| benzalkonium chloride | −4.02 | 0.18 |
| tetramethylammonium hexafluorophosphate | −3.58 | >0.1 |
| tetrabutylammonium hexafluorophosphate | −2.07 | >0.1 |
| ammonium hexafluorophosphate | −5.73 | 20 |

In the present invention, the Lewis acidity may be measured by reducing ubiquinone 1 using ubiquinone 1 (Q1), instead of oxygen molecule (02), to generate an anion radical of ubiquinone 1 in the Lewis acidity measuring method (1). In the following description, such a Lewis acidity measuring method may be referred to as the “Lewis acidity measuring method (2)”. In the Lewis acidity measuring method (2), the measurement can be performed in the same manner as in the Lewis acidity measuring method (1), except that ubiquinone 1 (Q1) is used instead of oxygen molecule (O₂). In the Lewis acidity measuring method (2), similarly to the Lewis acidity measuring method (1), the ΔE value (eV), which is an index of Lewis acidity, can be calculated from the obtained reaction rate constant (k_{cat}). The Lewis acidity measuring method (2) is, for example, described in Ohkubo, K.; Fukuzumi, S. Chem. Eur. J., 2000, 6, 4532, which is herein incorporated by reference, and can be performed according to or based on the method described therein.

The Lewis acidity measuring method (2) can be performed by measuring the reaction rate constant (k_{cat}) with respect to the following chemical reaction formula (1b).

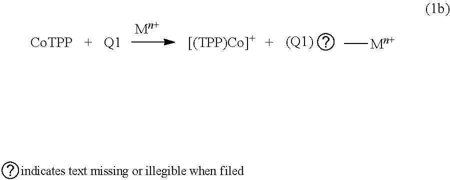

in the chemical formula (1b),
Mⁿ⁺ represents the radical generating catalyst,
CoTPP represents cobalt (II) tetraphenylporphyrin,
Q1 represents ubiquinone 1,
[(TPP)Co]⁺ represents cobalt (III) tetraphenylporphyrin cation, and
(Q1)^{⋅-} represents an anionic radical of ubiquinone 1.

The Lewis acidity of the radical generating catalyst of the present invention may have a reaction rate constant (k_{cat}) for the chemical reaction formula (1b), i.e., a measured value (K_{obs}) of the reaction rate constant (k_{cat}) measured by the “Lewis acidity measuring method (2)”, of, for example, 1.0×10⁻⁵ S⁻¹ or more, 2.0×10⁻⁵ S⁻¹ or more, 3.0×10⁻⁵ S⁻¹ or more, 4.0×10⁻⁵ S⁻¹ or more, 5.0×10⁻⁵ S⁻¹ or more, 6.0×10⁻⁵ S⁻¹ or more, 7.0×10⁻⁵ S⁻¹ or more, 8.0×10⁻⁵ S⁻¹ or more, 9.0×10⁻⁵ S⁻¹ or more, 1.0×10⁻⁴ S⁻¹ or more, 2.0×10⁻⁴ S⁻¹ or more, 3.0×10⁻⁴ S⁻¹ or more, 4.0×10⁻⁴ S⁻¹ or more, 5.0×10⁻⁴ S⁻¹ or more, 6.0×10⁻⁴ S⁻¹ or more, 7.0×10⁻⁴ S⁻¹ or more, 8.0×10⁻⁴ S⁻¹ or more, 9.0×10⁻⁴ S⁻¹ or more, 1.0×10⁻³ S⁻¹ or more, 2.0×10⁻³ S⁻¹ or more, 3.0×10⁻³ S⁻¹ or more, 4.0×10⁻³ S⁻¹ or more, 5.0×10⁻³ S⁻¹ or more, 6.0×10⁻³ S⁻¹ or more, 7.0×10⁻³ S⁻¹ or more, 8.0×10⁻³ S⁻¹ or more, 9.0×10⁻³ S⁻¹ or more, 1.0×10⁻² S⁻¹ or more, 2.0×10⁻² S⁻¹ or more, 3.0×10⁻² S⁻¹ or more, 4.0×10⁻² S⁻¹ or more, 5.0×10⁻² S⁻¹ or more, 6.0×10⁻² S⁻¹ or more, 7.0×10⁻² S⁻¹ or more, 8.0×10⁻² S⁻¹ or more, or 9.0×10⁻² S⁻¹ or more; or 1.0×10⁻¹ S⁻¹ or less, 9.0×10⁻² S⁻¹ or less, 8.0×10⁻² S⁻¹ or less, 7.0×10⁻² S⁻¹ or less, 6.0×10⁻² S⁻¹ or less, 5.0×10⁻² S⁻¹ or less, 4.0×10⁻² S⁻¹ or less, 3.0×10⁻² S⁻¹ or less, 2.0×10⁻² S⁻¹ or less, 1.0×10⁻² S⁻¹ or less, 9.0×10⁻³ S⁻¹ or less, 8.0×10⁻³ S⁻¹ or less, 7.0×10⁻³ S⁻¹ or less, 6.0×10⁻³ S⁻¹ or less, 5.0×10⁻³ S⁻¹ or less, 4.0×10⁻³ S⁻¹ or less, 3.0×10⁻³ S⁻¹ or less, 2.0×10⁻³ S⁻¹ or less, 1.0×10⁻³ S⁻¹ or less, 9.0×10⁻⁴ S⁻¹ or less, 8.0×10⁻⁴ S⁻¹ or less, 7.0×10⁻⁴ S⁻¹ or less, 6.0×10⁻⁴ S⁻¹ or less, 5.0×10⁻⁴ S⁻¹ or less, 4.0×10⁻⁴ S⁻¹ or less, 3.0×10⁻⁴ S⁻¹ or less, 2.0×10⁻⁴ S⁻¹ or less, 1.0×10⁻⁴ S⁻¹ or less, 9.0×10⁻⁵ S⁻¹ or less, 8.0×10⁻⁵ S⁻¹ or less, or 7.0×10⁻⁵ S⁻¹ or less.

In the radical generating catalyst of the present invention, the ammonium may be, for example, quaternary ammonium, or tertiary, secondary, primary, or zero ammonium. The ammonium is not particularly limited, and may be, for example, a nucleic acid base or the like, or an amino acid, a peptide, or the like described below.

The radical generating catalyst of the present invention may be, for example, a cationic surfactant, which may be a quaternary ammonium-type cationic surfactant. Examples of the quaternary ammonium-type cationic surfactant include benzalkonium chloride, benzethonium chloride, cetylpyridinium chloride, hexadecyltrimethylammonium bromide, dequalinium chloride, edrophonium, didecyldimethylammonium chloride, tetramethylammonium chloride, tetrabutylammonium chloride, benzyltriethylammonium chloride, oxytropium, carbachol, glycopyrronium, safranin, sinapine, tetraethylammonium bromide, hexadecyltrimethylammonium bromide, suxamethonium, sphingomyelin, ganglioside GM1, denatonium, trigonelline, neostigmine, paraquat, pyridostigmine, phellodendrine, pralidoxime methiodide, betaine, betanin, bethanechol, betalain, lecithin, adenine, guanine, cytosine, thymine, uracil, and cholines (e.g., choline chlorides [such as benzoyl choline chloride and a lauroylcholine chloride hydrate], phosphocholine, acetylcholine, choline, dipalmitoylphosphatidylcholine, and choline bitartrate). It is to be noted, however, that, in the radical production method for the present invention, the quaternary ammonium is not limited to a surfactant.

In the radical generating catalyst of the present invention, the ammonium may be for example, an ammonium salt represented by the following chemical formula (XI).

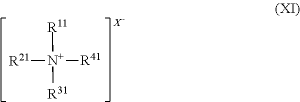

In the chemical formula (XI), R¹¹, R²¹, R³¹, and R⁴¹ are each a hydrogen atom or an aromatic ring, or an alkyl group, and the alkyl group may include an ether bond, a carbonyl group, an ester bond, or an amide bond, or an aromatic ring, and R¹¹, R²¹, R³¹, and R⁴¹ may be identical to or different from each other, or two or more of R¹¹, R²¹, R³¹, and R⁴¹ may be integrated to form a ring structure with N⁺ to which they are bonded, and the ring structure may be saturated or unsaturated, aromatic or non-aromatic, and may or may not have one or more substituents, and X⁻ is an anion, and X⁻ is, for example, an anion excluding peroxodisulfate ion. In R¹¹, R²¹, R³¹, and R⁴¹, the aromatic ring is not particularly limited, and for example, may or may not contain a heteroatom, and may or may not have a substituent. Examples of the aromatic ring containing a heteroatom (heteroaromatic ring) include a nitrogen-containing aromatic ring, a sulfur-containing aromatic ring, and an oxygen aromatic ring. Examples of the aromatic ring not containing a heteroatom include a benzene ring, a naphthalene ring, an anthracene ring, and a phenanthrene ring. Examples of the heteroaromatic ring include a pyridine ring, a thiophene ring, and a pyrene ring. The nitrogen-containing aromatic ring, for example, may or may not have a positive charge=. Examples of the nitrogen-containing aromatic ring having no positive charge include a pyrroline ring, a pyridine ring, a pyridazine ring, a pyrimidine ring, a pyrazine ring, a quinoline ring, an isoquinoline ring, an acridine ring, a 3,4-benzoquinoline ring, a 5,6-benzoquinoline ring, a 6,7-benzoquinoline ring, a 7,8-benzoquinoline ring, a 3,4-benzoisoquinoline ring, a 5,6-benzoisoquinoline ring, a 6,7-benzoisoquinoline ring, and a 7,8-benzoisoquinoline ring. Examples of the nitrogen-containing aromatic ring having a positive charge include a pyrrolinium ring, a pyridinium ring, a pyridazinium ring, a pyrimidinium ring, a pyrazinium ring, a quinolinium ring, an isoquinolinium ring, an acridinium ring, a 3,4-benzoquinolinium ring, a 5,6-benzoquinolinium ring, a 6,7-benzoquinolinium ring, a 7,8-benzoquinolinium ring, a 3,4-benzoisoquinolinium ring, a 5,6-benzoisoquinolinium ring, a 6,7-benzoisoquinolinium ring, and a 7,8-benzoisoquinolinium ring. The oxygen-containing aromatic ring or the sulfur-containing aromatic ring may be, for example, an aromatic ring in which at least one of a carbon atom or a nitrogen atom of the above-described heteroatom-free aromatic ring or nitrogen-containing aromatic ring is substituted with at least one of an oxygen atom or a sulfur atom. In R¹¹, R²¹, R³¹, and R⁴¹, when the alkyl group or the aromatic ring has a substituent, the substituent is not particularly limited and is optional, and examples thereof include a sulfo group, a nitro group, and a diazo group.

The ammonium salt represented by the chemical formula (XI) may be, for example, an ammonium salt represented by the following chemical formula (XII).

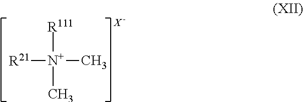

In the chemical formula (XII), R¹¹¹ is an alkyl group having 5 to 40 carbon atoms and may include an ether bond, a ketone (carbonyl group), an ester bond, or an amide bond, substituent, or an aromatic ring, and R²¹ and X⁻ are the same as those in the chemical formula (XI). In R¹¹¹, the aromatic ring is not particularly limited, and, for example may or may not contain a heteroatom, and may or may not have a substituent. In R¹¹¹, the aromatic ring is not particularly limited, and specific examples are the same as those in R¹¹, R²¹, R³¹, and R⁴¹ of the chemical formula (XI). In R¹¹¹, when the alkyl group or the aromatic ring has a substituent, the substituent is not particularly limited, is optional, and is, for example, the same as those in R¹¹, R²¹, R³¹, and R⁴¹ of the chemical formula (XI).

In the chemical formula (XII), R²¹ may be, for example, a methyl group or a benzyl group. In the benzyl group, one or more hydrogen atoms of the benzene ring may or may not be substituted with any substituent. The substituent may be, for example, an alkyl group, an unsaturated aliphatic hydrocarbon group, an aryl group, a heteroaryl group, a halogen, a hydroxy group (—OH), a mercapto group (—SH), or an alkylthio group (—SR, where R is an alkyl group).

The ammonium salt represented by the chemical formula (XII) may be, for example, an ammonium salt represented by the following chemical formula (XIII).

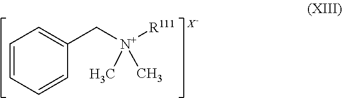

In the chemical formula (XIII), R¹¹¹ and X⁻ are the same as those in the chemical formula (XII).

The ammonium may be, for example, at least one material selected from the group consisting of benzethonium chloride, benzalkonium chloride, hexadecyltrimethylammonium chloride, tetramethylammonium chloride, ammonium chloride, methylammonium chloride, tetrabutylammonium chloride, cetylpyridinium chloride, hexadecyltrimethylammonium bromide, dequalinium chloride, edrophonium, didecyldimethylammonium chloride, benzyltriethylammonium chloride, oxytropium, carbachol, glycopyrronium, safranin, sinapine, tetraethylammonium bromide, hexadecyltrimethylammonium bromide, suxamethonium, sphingomyelin, ganglioside GM1, denatonium, trigonelline, neostigmine, paraquat, pyridostigmine, phellodendrine, pralidoxime methiodide, betaine, betanin, bethanechol, betalain, lecithin, adenine, guanine, cytosine, thymine, uracil, and cholines. The ammonium may be, for example, benzethonium chloride.

The ammonium salt represented by the chemical formula (XI) may be, for example, an ammonium salt represented by the following chemical formula (XIV).

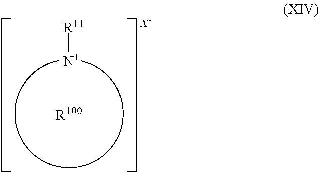

Where in the chemical formula (XIV), R¹⁰⁰ may form a ring structure, which may be saturated or unsaturated, aromatic or non-aromatic, and may or may not have one or more substituents, and R¹¹ and X⁻ are the same as those in the chemical formula (XI).

The ammonium salt represented by the chemical formula (XI) may be, for example, an ammonium salt represented by the following chemical formula (XV).

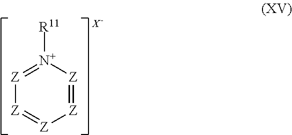

In the chemical formula (XV), Zs are each CH or N, may be identical to or different from each other, and in the case of CH, H may be substituted with a substituent, and R¹¹ and X⁻ are the same as those in the chemical formula (XI).

The ammonium salt represented by the chemical formula (XI) may be, for example, an ammonium salt represented by the following chemical formula (XVI).

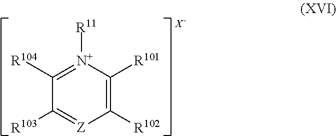

In the chemical formula (XVI), R¹⁰¹, R¹⁰², R¹⁰³, and R¹⁰⁴ are each a hydrogen atom or a substituent, and R¹⁰¹, R¹⁰², R¹⁰³, and R¹⁰⁴ may be identical to or different from each other, or two or more of R¹⁰¹, R¹⁰², R¹⁰³, and R¹⁰⁴ may be integrated to form a ring structure with N⁺ to which they are bonded, and the ring structure may be saturated or unsaturated, and aromatic or non-aromatic, and may or may not have one or more substituents, Z is CH or N, and in the case of CH, H may be substituted with a substituent, and R¹¹ and X⁻ are the same as those in the chemical formula (XI).

The ammonium salt represented by the chemical formula (XI) may be, for example, an ammonium salt represented by the following chemical formula (XVII).

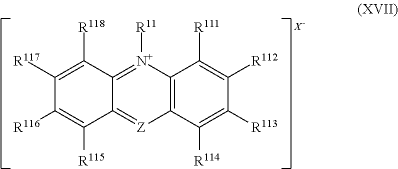

In the chemical formula (XVII), R¹¹¹ to R¹¹⁸ are each a hydrogen atom or a substituent, and may be identical to or different from each other, or two or more of R¹¹¹ to R¹¹⁸ may be integrated to form a ring structure, which may be aromatic or non-aromatic and may or may not have one or more substituents, Z is CH or N, and in the case of CH, H may be substituted with a substituent, and R¹¹ and X⁻ are the same as those in the chemical formula (XI).

The ammonium salt represented by the chemical formula (XI) may be, for example, at least one selected from the group consisting of benzethonium chloride, benzalkonium chloride, hexadecyltrimethylammonium chloride, tetramethylammonium chloride, ammonium chloride, methylammonium chloride, and tetrabutylammonium chloride. It is particularly preferable that the ammonium salt represented by the chemical formula (XII) is benzalkonium chloride.

Benzethonium chloride (Bzn⁺Cl⁻) can be, for example, represented by the following chemical formula. Benzalkonium chloride can be, for example, a compound represented by the chemical formula (XIII) where R¹¹¹ is an alkyl group having 8 to 18 carbon atoms and X⁻ is a chloride ion.

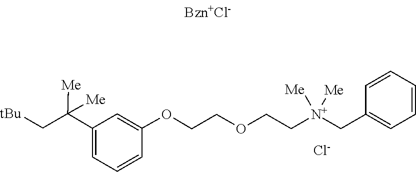 [SMILES: CC(C)(C)CC(C)(C)c1cccc(OCCOCC[N+](C)(C)Cc2ccccc2)c1 ; [Cl-]]

In the chemical formulae (XI), (XII), (XIII), (XIV), (XV), (XVI), and (XVII), X⁻ may be any anion and is not particularly limited. X⁻ is not limited to a monovalent anion, and may be an anion with any valence, such as a divalent anion or a trivalent anion. When the anion is an anion with a plurality of electric charges, such as a divalent anion or a trivalent anion, the number of molecules of the (monovalent) ammonium in each of the chemical formulae (XI), (XII), (XIII), (XIV), (XV), (XVI), and (XVII) is determined by, for example, the number of molecules of the anion x the valence of the anion (e.g., when the anion is divalent, the number of molecules of the (monovalent) ammonium is twice the number of molecules of the anion). X⁻ may be, for example, a halogen ion (a fluoride ion, a chloride ion, a bromide ion, or an iodide ion), an acetate ion, a nitrate ion, or a sulfate ion.

In the present invention, the radical generating catalyst is not limited to the chemical formulae (XI), (XII), (XIII), (XIV), (XV), (XVI) and (XVII), and may be ammonium having any structure containing an aromatic ring. The aromatic ring is not particularly limited, and may be, for example, an aromatic ring exemplified in R¹¹, R²¹, R³¹, and R⁴¹ of the chemical formula (XI).

In the present invention, the radical generating catalyst may be, for example, a sulfonic acid amine or ammonium thereof. The sulfonic acid amine is, for example, amine having a sulfonic group (sulfonic acid group) in its molecule. Examples of the sulfonic acid amine include taurine, sulfamic acid, 3-amino-4-hydroxy-1-naphthalenesulfonic acid, sulfamic acid, p-toluidine-2-sulfonic acid, o-anisidine-5-sulfonic acid, direct blue 14, 3-[N, N-bis (2-hydroxyethyl)amino]-2-hydroxypropanesulfonic acid, 3-[(3-colamidopropyl)dimethylammonio]-1-propanesulfonate, aminomethanesulfonic acid, 3-sulfopropylamine, 2-aminobenzenesulfonic acid, R(+)-3-aminotetrahydrofuran, toluene, 4-amino-5-hydroxy-1,7-naphthalenedisulfonic acid, N-(2-acetamido)-2-aminoethanesulfonic acid, 4′-amino-3′-methoxyazobenzene-3-sodium sulfonate, Lapatinib ditosylate, N-tris (hydroxymethyl) methyl-2-aminoethanesulfonic acid, 8-amino-1,3,6-naphthalenetrisulfonic acid disodium hydrate, 1-aminonaphthalene-2-sulfonic acid, (2S,3S)-3-Amino-2-methyl-4-oxo-1-azetidinesulfonic acid, sodium 3-(1-naphthylamino) propanesulfonate, 3-methyl-4-aminobenzenesulfonic acid, sodium 3-Cyclohexylamino-2-hydroxypropanesulfonic acid, sodium N-tris (hydroxymethyl) methyl-2-aminoethanesulfonic acid, 4-amino-1-naphthalenesulfonic acid, sodium sulfamate, tricaine, sodium sulfanilate, 1,4-phenylenediamine 2-sulfonic acid, p-anisidine-2-sulfonic acid, 6-amino-1-naphthalenesulfonic acid, 3,4-diaminobenzene sulfonic acid, 3-amino-4-chlorobenzene sulfonic acid, 3-[(4-Amino-3-methylphenyl) azo] benzenesulfonic acid, 3-amino-4-hydroxy-5-nitrobenzenesulfonic acid, 5-amino-6-hydroxy-3-nitrobenzenesulfonic acid, 4-acetamide-2-Aminobenzenesulfonic acid hydrate, 2-aminophenol-4-sulfonic acid, 1-amino-2-methoxy-5-methyl-4-benzenesulfonic acid, dansylic acid, Sulfamic acid [(1S, 2S, 4R)-4-[4-[[(1S)-2,3-dihydro-1H-inden-1-yl] amino]-7H-pyrrolo [2,3-d] pyrimidin-7-yl]-2-hydroxycyclopropyl] methyl ester, 5-sulfo-4′-diethylamino-2,2′ dihydroxyazobenzene, 2-aminonaphthalene-6,8-disulfonic acid, sodium 2-[N,N-bis (2-hydroxyethyl) amino]-1-ethanesulfonate, 3-acetyl-2-(methylaminosulfonyl) thiophene, sodium 4-amino-2-chlorotoluene-5-sulfonate, 5-(3-amino-5-oxo-2-pyrazolin-1-yl)-2-phenoxybenzenesulfonic acid, potassium sulfamate, p-aminoazobenzene monosulfonic acid, 3-[(3-Cholamidopropyl) dimethylamino]-2-hydroxy-1-propanesulfonate, 3-amino-2,7-naphthalenes disulfonic acid monosodium, 3-[N, N-bis (hydroxyethyl) amino]-2-hydroxypropanesulfonic acid sodium salt, di (amidosulfuric acid) cobalt (II), 3-(4-amino-3-methoxyphenylazo) benzenesulfonic acid, Nickel (II) sulfamate tetrahydrate, sodium 2,4-diaminobenzenesulfonate, 5-amino-2-chlorotoluene-4-sulfonic acid, 2,5-dichlorosulfanilic acid, 4-methylbenzenesulfonic acid, APTS (aminopyrenetrisulfonic acid), 4′-aminoazobenzene-3-sulfonic acid, Pontacyl carmine 2B, p-anisidine-3-sulfonic acid, 4,4′-bis (4-amino-1-naphthylazo)-2,2′-stilbenesulfonic acid, 3-aminonaphthalene-8-hydroxy-4, 6-disulfonic acid, sodium 4-amino-1,5-naphthalenedisulfonate, sodium 4-aminoazobenzene-4′-sulfonate, 5-amino-2-methylbenzenesulfonic acid, disodium 7-amino-1,3-naphthalene disulfonate, alizarin safilol SE, sodium 7-amino-2-naphthalenesulfonate, 6-amino-5-bromopyridine-3-sulfonic acid, 2-aminoethanethiol p-toluenesulfonate, sodium 2-amino 1-naphthalenesulfonate, 6-amino-1,3-naphthalenedisulfonic acid disodium hydrate, N,N,N,N′-tetraethylsulfamide, 5-amino-2-ethoxybenzenesulfonic acid, 3,5-diamino-2,4,6-trimethylbenzenes phosphonic acid, 7-amino-1-naphthalenesulfonic acid, sulfamic acid, guanidine, 2-amino-5-nitrobenzenesulfonic acid, nickel (II) diamide sulfate, 4-amino-4′-nitrostilbene-2,2′-disulfonic acid disodium, aniline-2,5-disulfonic acid monosodium, 5-amino-1-naphthol-3-sulfonic acid hydrate, 2,5-dichlorosulfanilic acid sodium salt, 6-aminohexanoic acid hexyl p-toluenesulfonate, rac-(R*)-2-(4-chlorophenyl)-3-amino-1-propanesulfonic acid, 2-(N,N-dipropyl) amino anisole-4-Sulfonic acid, 2-amino-4-chlorophenol-6-sulfonic acid, 6-amino-1,3-naphthalenedisulfonic acid, 5,10,15,20-tetrakis [4-(trimethylammonio) phenyl]-21H,-23H-porphine tetratosylate, 5-amino-2-[(4-aminophenyl) amino] benzenesulfonic acid, 4-amino-3-chlorobenzenesulfonic acid, 2-aminobenzenesulfonic acid phenyl ester, 4-Acetylamino-4′-isothiocyanatostilbene-2,2′-disulfonic acid disodium salt, (S)-3-amino-2-oxetanone p-toluenesulfonic acid salt, 5-acetylamino-4-hydroxy-2,7-naphthalenedisulfonic acid disodium salt, 2-phenylamino-5-aminobenzenesulfonic acid, sodium 4-octadecylamino-4-oxo-2-[(sodiooxy)sulfonyl]butanoate, and 3,5-diamino-4-methylbenzenesulfonic acid.

In the present invention, the radical generating catalyst may be, for example, nicotinic amine or ammonium thereof. The nicotinic amine is, for example, an amine having a ring structure in a molecule, and the ring structure has a nicotine skeleton. Examples of the nicotinic amine include nicotinamide and alkaloid.

In the present invention, the radical generating catalyst may be, for example, nitrite amine or nitrite ammonium. The nitrite amine or nitrite ammonium is, for example, a compound obtained by reacting amine with nitrous acid or a nitrous acid derivative. Examples of the nitrite amine or nitrite ammonium include diazo compounds, diazonium salts, N-nitroso compounds, and C-nitroso compounds.

In the radical generating catalyst of the present invention, the ammonium may include a plurality of ammonium structures (N⁺) in one molecule. Further, the ammonium may, for example, form a dimer, trimer, or the like by association of a plurality of molecules through a π electron interaction.

In the radical generating catalyst of the present invention, the amino acid is not particularly limited. The amino acid may contain, for example, at least one of both an amino group or an imino group, or a carboxy group in the molecule. The amino acid may be, for example, an α-amino acid, a β-amino acid, a γ-amino acid, or any other amino acid. The amino acid may be, for example, an amino acid constituting protein, and specifically may be at least one selected from the group consisting of, for example, glycine, alanine, valine, leucine, isoleucine, serine, threonine, aspartic acid, glutamic acid, asparagine, glutamine, lysine, hydroxylysine, arginine, cysteine, cystine, methionine, phenylalanine, tyrosine, tryptophan, histidine, proline, and 4-hydroxyproline.

In the radical generating catalyst of the present invention, the peptide is not particularly limited. The peptide may be, for example, one in which two or more of the amino acid molecules are linked by a peptide bond. The peptide may be, for example, at least one of oxidized glutathione (GSSG) or reduced glutathione (GSH).

In the radical generating catalyst of the present invention, the phospholipid is not particularly limited. The phospholipid may be, for example, a lipid containing phosphorus atoms in the molecule, and may be, for example, a lipid containing a phosphate ester bond (P—O—C) in the molecule. The phospholipid may or may not have, for example, at least one of an amino group, an imino group, an ammonium group, or an iminium group, in the molecule. The phospholipid may be, for example, at least one selected from the group consisting of phosphatidylserine, phosphatidylcholine, phosphatidic acid, phosphatidylethanolamine, phosphatidylglycerol, and cardiolipin.

The content of the radical generating catalyst of the present invention in the oral care agent of the present invention is not particularly limited, and may be, for example, 0.0001% by mass or more, 0.001% by mass or more, 0.01% by mass or more, 0.1% by mass or more, or 0.2% by mass or more, and may be, for example, 10% by mass or less, 8% by mass or less, 5% by mass or less, 3% by mass or less, 2% by mass or less, 1% by mass or less, 0.5% by mass or less, 0.3% by mass or less, or 0.2% by mass or less with respect to the total mass of the oral care agent of the present invention. One type of radical generating catalysts of the present invention may be used alone or two or more types of them may be used in combination.

The oral care agent of the present invention may or may not include components other than the base, the antimicrobial component, the antiviral component, the radical generator, and the radical generating catalyst. Examples of the other component include, but are not limited to, a pH adjuster, a corrigent, a sweetener, a flavoring agent, a flavoring and seasoning agent, and the like, and one type the component may be used alone or two or more types of the component may be used in combination. The other component may be, for example, components listed as examples of the component of the dentifrice to be described below. The content of the other components in the oral care agent of the present invention is not particularly limited, and may be, for example, 0.001% by mass or more, 0.01% by mass or more, 0.1% by mass or more, 1% by mass or more, or 2% by mass or more, and may be, for example, 10% by mass or less, 8% by mass or less, 5% by mass or less, 3% by mass or less, or 2% by mass or less with respect to the total mass of the oral care agent of the present invention.

The oral care agent of the present invention may also be, for example, a dentifrice. By using the oral care agent of the present invention as a dentifrice, it is possible to effectively remove, for example, oral deposits adhered to gingivae or teeth, and also to treat or prevent, for example, caries, periodontal disease, and the like. The oral care agent of the present invention which is a dentifrice (hereinafter, also referred to as the “dentifrice of the present invention”) may or may not include the base, the antimicrobial component, the antiviral component, the radical generator, or the radical generating catalyst. The dentifrice of the present invention may or may not include components other than the base, the antimicrobial component, the antiviral component, the radical generator, and the radical generating catalyst. The other components are not particularly limited, and may be, for example, the same as the components of a general dentifrice. The content of the other components is not particularly limited, and may be, for example, the same as or similar to that of a general dentifrice. Examples of the other component in the dentifrice of the present invention include an abrasive, a humectant, sugar alcohol, a foaming agent, a binder, a flavoring and seasoning agent, a preservative, a cleaning agent, a viscosity modifier, a pH adjuster, a stabilizer, and a medicinal component. The abrasive serves, for example, to remove plaque or stain without damaging the tooth surface. Examples of the abrasive include calcium hydrogen phosphate, aluminum hydroxide, and silicic anhydride. The humectant gives moisture to, for example, toothpaste. Examples of the humectant include polyhydric alcohols such as sorbitol (sorbitol solution), glycerin, and propylene glycol. The sugar alcohol serves, for example, as a thickener, and examples thereof include xylitol, sorbitol, carboxymethylcellulose (CMC), and hydroxyethylcellulose (HEC). The foaming agent serves, for example, to diffuse the toothpaste with foam and wash the inside the mouth, and examples thereof include sodium lauryl sulfate and coconut oil fatty acid amidopropyl betaine. The binder serves, for example, to bind and retain the components of the toothpaste, and examples thereof include sodium carboxymethyl cellulose, sodium alginate, and xanthan gingiva. Examples of the viscosity modifier include acetic anhydride silicic acid and sodium polyacrylate. The pH adjuster may be, for example, sodium hydroxide. The stabilizer serves, for example, to suppress separation or the like of components of the dentifrice, and examples thereof include titanium oxide. The flavoring and seasoning agent serves, for example, to flavor or season, and examples thereof include a flavoring agent (e.g., a double mint flavor, a mild pure mint flavor, etc.), xylitol, saccharin, sodium saccharin, menthol, and mint. The preservative serves, for example, as an antisepsis and examples thereof include paraben and sodium benzoate. Examples of the cleaning agent include hydrous silicic acid and acetic anhydride silicic acid A. Examples of the medicinal component include fluoride, an anti-inflammatory agent, a bactericide, a pain stimulant blocking agent, a stain remover, and a repair agent. The fluoride serves, for example, to promote remineralization of teeth or to improve acid resistance of teeth, and examples thereof include sodium fluoride and sodium monofluorophosphate. The anti-inflammatory agent serves, for example, to sedate inflammation, prevent bleeding, and reduce inflammation of teeth or gingivae, and examples thereof include tranexamic acid, E-aminocaproic acid, p-glycyrrhizic acid, and phellodendron bark extract. Examples of the bactericide (bacteriocidal component) include the above-mentioned radical generator, isopropylmethylphenol (IPMP, sold under the trade name “Biozole” by Osaka Kasei Co., Ltd.), cetylpyridinium chloride, and ethanol. The pain stimulant blocking agent serves, for example, to suppress sensory hypersensitivity by suppressing transmission of stimulation to the nerve of the tooth, and examples thereof include aluminum lactate and potassium nitrate. The stain remover serves to promote the dissolution of stain, and may be, for example, polyethylene glycol. The repair agent serves, for example, to repair damage of teeth, and may be, for example, hydroxyapatite.

[2. Production Method, Use Method, and the Like of Oral Care Agent]

The method for producing an oral care agent of the present invention is not particularly limited, and may be, for example, simply mixing all the components of the oral care agent of the present invention.

The method for using the oral care agent of the present invention is not particularly limited, and the oral care agent of the present invention may be used, for example, by applying to the oral cavity. The site of the oral cavity to be applied with the oral care agent is not particularly limited, and the oral care agent may be applied, for example, to the entire or only a part of the oral cavity. The oral care agent-applied site may be, for example, an oral mucosa, a tongue, or the like. When applied to the oral mucosa, the oral care agent may be applied to the entire or a part of the oral mucosa, for example, the oral care agent may be applied to the palatal side, lingual side, or labial side of the gingiva, the maxilla, the mandible, or the like. In the case of application to the tongue, the oral care agent may be applied to the entire or a part of the tongue, for example, to one or both of the front and back surfaces of the tongue. The oral care agent of the present invention may be applied to the entire oral cavity, or may be applied only to a site where oral deposits are adhered, for example, as described above. The oral deposits may be, but not limited to, at least one substance selected from the group consisting of sputum, crust, exfoliated epithelium, clot, bacteria, virus, dental plaque, and biofilm, as described above. The biofilm may be, but not limited to, a biofilm formed from one or more types of oral deposits such as sputum, crust, exfoliated epithelium, clot, bacteria, virus, dental plaque, and the like, as described above.

The application amount of the oral care agent of the present invention is not particularly limited, and may be, for example, 10 mg or more, 20 mg or more, 30 mg or more, 40 mg or more, or 50 mg or more per cm² of oral care agent-applied site per application, and, for example, 50 mg or less, 40 mg or less, 30 mg or less, or 10 mg or less per cm² of oral care agent-applied site per application. The application interval is not particularly limited, and may be, for example, 1 time or more, 2 times or more, 3 times or more, 4 times or more, or 5 times or more a week, or 1 time or more, 2 times or more, 3 times or more, 4 times or more, or 5 times or more a day, and may be, for example, 5 times or less, 4 times or less, 3 times or less, 2 times or less, 1 time or less a week, or 5 times or less, 4 times or less, 3 times or less, 2 times or less, 1 time or less a day. The period during which the application is continued is not particularly limited, and may be, for example, 1 day or more, 3 days or more, 7 days or more, 30 days or more, or 365 days or more, and may be, for example, 365 days or less, 30 days or less, 7 days or less, 3 days or less, or 1 day or less.

The patient to which the oral care agent of the present invention is to be administered is, for example, a human or a non-human animal excluding a human. Examples of a non-human animal include mice, rats, rabbits, monkeys, pigs, dogs, cows, horses, and cats.

The oral care agent of the present invention can be applied to the oral cavity by being semi-solid, for example, and can remain on the applied surface without flowing down therefrom. As a result, for example, it is possible to soften the oral deposits adhered to the surface of the oral cavity. The oral deposits can be, for example, thereby removed without damaging the oral surface. More specifically, for example, by softening dried and hardened sputum and exfoliated epithelium, clot, bacteria, virus, dental plaque, and biofilm of a human (or non-human animal) with the oral care agent of the present invention, it is possible to safely and quickly (efficiently) peel the oral deposits without damaging the oral mucosa. Not damaging the oral mucosa allows the damage, bleeding, pain, postoperative infection, and the like of the oral mucosa to be prevented. In addition, according to the oral care agent of the present invention, for example, it is possible to suppress or prevent re-adhesion of oral deposits (for example, sputum and exfoliated epithelium, clot, bacteria, virus, dental plaque, biofilm, and the like) to the oral mucosa, which makes it easier to provide continuous oral care.

It is presumed that the reason why the oral deposits can be softened by the oral care agent of the present invention is, for example, that the oral care agent of the present invention dissolves a blood component contained in the oral deposits (e.g., sputum, exfoliated epithelium, etc.) or a biofilm composed of an extracellular matrix. In addition, since the oral care agent of the present invention is semi-solid, for example, as described above, it is possible to remain on the applied surface without flowing down therefrom, and therefore, it is considered that the contact time with the oral deposits is prolonged and the oral deposits are easily softened. As described above, by softening the oral deposits, the oral deposits can be safely removed without damaging the surface of the oral cavity.

In addition, it is considered that since the oral care agent of the present invention includes a radical generator, for example, the radicals generated from the radical generator cause chemical change (e.g., decomposing proteins, etc.) of the oral deposits (e.g., sputum, exfoliated epithelium, etc.), whereby the oral deposits are easily peeled off from the surface of the oral cavity. Further, it is considered that since the oral care agent of the present invention includes a radical generator and is semi-solid, for example, the radical generator easily remains on the surface of the oral cavity and the effect of the radical generator is further promoted. Furthermore, it is considered that since the oral care agent of the present invention includes a radical generating catalyst, for example, the effect of the radical generator can further be promoted.

### EXAMPLES

Hereinafter, examples of the present invention will be described. The present invention, however, is not limited to the following examples.

### Example 1

### (1) Production of Oral Care Agent

Oral care agents were prepared by mixing the following compositions [1] to [30]. In the following description, water was used as a solvent or a dispersion medium, unless otherwise specified. In the following description, the % concentration is % by mass unless otherwise specified, and ppm concentration is also ppm by mass unless otherwise specified. In addition, in the following description, “MA-Ty” refers to the inclusion of sodium chlorite and benzalkonium chloride in a mass ratio of 5:3. For example, “MA-Ty 200 ppm” refers to the inclusion of 200 ppm sodium chlorite and 120 ppm benzalkonium chloride. In addition, the oral care agents of [1] to [30] below were prepared so as to include 5 mM Na₂HPO₄/NaH₂PO₄ in addition to the compositions below. Specifically, both of the following production method 1 and the following production method 2 were employed, and the oral care agents could be produced and used by either of the production method 1 or the production method 2. As described in [1] to [30] below, xylitol was used as a thickener alone or in combination with other sugar alcohols. While sugar alcohols other than xylitol had taste, sweetness, or viscosity enhancing effects, those using xylitol alone (without the addition of other sugar alcohols) were the most difficult to react with sodium chlorite and benzalkonium chloride and were stable.

[1]
200 ppm MA-Tγ
2% carboxymethylcellulose (CMC)
1% xylitol
   [2]
(Soft Type)
200 ppm MA-Tγ
1% CMC
1% xylitol
   [3]
200 ppm MA-Tγ
3% hyaluronic acid
1% xylitol
   [4]
200 ppm MA-Tγ
2% hyaluronic acid
1% xylitol
   [5]
200 ppm MA-Tγ
2% hydroxyethylcellulose (HEC)
1% xylitol
   [6]
200 ppm MA-Tγ
3% HEC
1% xylitol
   [7]
200 ppm MA-Tγ
0.1% hyaluronic acid
1% xylitol
   [8]
200 ppm MA-Tγ
2% CMC (trade name “CEKOL100000” (high-viscosity CMC), Sansho Co., Ltd.)
1% xylitol
   [9]
200 ppm MA-Tγ
0.5% CMC
1% xylitol
   [10]
200 ppm MA-Tγ
1.0% CMC (trade named: “CEKOL100000” (high-viscosity CMC), Sansho Co., Ltd.)
1% xylitol
   [11]
200 ppm MA-Tγ
1.25% CMC
1% xylitol
   [12]
200 ppm MA-Tγ
1.5% CMC
1% xylitol
   [13]
200 ppm MA-Tγ
1.5% hyaluronic acid
1% xylitol
   [14]
200 ppm MA-Tγ
0.75% CMC
1% xylitol
   [15]
200 ppm MA-Tγ
1.5% hyaluronic acid
1% xylitol
   [16]
200 ppm MA-Tγ
3% HEC
1% xylitol
   [17]
200 ppm MA-Tγ
3% HEC
0.25% xylitol
20% glycerin
   [18]
200 ppm MA-Tγ
2.5% HEC
0.25% xylitol
20% glycerin
   [19]
200 ppm MA-Tγ
2% CMC
10% glycerin
0.125% xylitol
   [20]
Prepared by the following production method 1 (separately mixed)
200 ppm MA-Tγ
1.5% HEC
1% xylitol
1% sorbitol
   [21]
Prepared by the following production method 2 (mixing at once)
200 ppm MA-Tγ
1.5% HEC
1% xylitol
1% sorbitol
   [22]
200 ppm MA-Tγ
1.5% CMC independent sonication
1% xylitol
   [23]
200 ppm MA-Tγ
1.5% HEC
1% xylitol
1% sorbitol
post-mixing sonication
   [24]
200 ppm MA-Tγ
1.5% CMC independent sonication
1% xylitol
1% sorbitol
   [25]
200 ppm MA-Tγ
2% HEC independent sonication
0.5% xylitol
0.5% sorbitol
   [26]
200 ppm MA-Tγ
1.5% CMC independent sonication
0.5% xylitol
0.5% sorbitol
   [27]
200 ppm MA-Tγ
2% HEC independent sonication
0.5% xylitol
0.5% sorbitol
post-mixing sonication
   [28]
200 ppm MA-Tγ
2% HEC independent sonication
1% xylitol
0.5% sorbitol
No sonication after mixing
   [29]
100 ppm MA-Tγ
1.5% CMC
1% xylitol
post-mixing sonication
   [30]
100 ppm MA-Tγ
2% HEC
1% xylitol
post-mixing sonication

[Production Method 1]

40000 ppm MA-Tγ was diluted with purified water to produce 400 ppm MA-Tγ. To 50 ml of this 400 ppm MA-Tγ, xylitol was added so that the concentration (final concentration) in the oral care agent was 1% by mass, and further, Na₂HPO₄/NaH₂PO₄ was added so that the concentration (final concentration) in the oral care agent was 5 mM. The obtained solution was used as solution A. On the other hand, a thickener other than xylitol was added to 50 ml of purified water at a predetermined concentration as necessary. The obtained solution was used as solution B. When a thickener other than xylitol was not used, 50 ml of only the purified water was used as solution B. The solution A and the solution B were mixed, and the mixture was thoroughly stirred using a homogenizer as necessary to prepare an oral care agent.

[Production Method 2]

40000 ppm MA-Tγ was diluted with purified water to produce 400 ppm MA-Tγ. To 1000 ml of this 400 ppm MA-Tγ, xylitol was added so that the concentration (final concentration) in the oral care agent was 1% by mass, and further, Na₂HPO₄/NaH₂PO₄ was added so that the concentration (final concentration) in the oral care agent was 5 mM. A thickener other than xylitol was added thereto and mixed at a predetermined concentration as necessary, and the mixture was thoroughly stirred using a homogenizer as necessary to prepare an oral care agent. When a thickener other than xylitol was not used, the other thickener was not added.

### (2) Removal of Oral Deposits by Oral Care Agent

The oral care agent prepared in (1) above was applied to the entire oral cavity including the oral mucosa and tongue of the patient. The oral care agent-applied site includes the entire mucosa in the oral cavity, and specifically includes the lingual and labial sides of the gingivae, the maxilla, the mandible, and the like. In addition, the oral care agent-applied site includes the entire tongue, and specifically includes the front and back surfaces of the tongue. The amount of the oral care agent to be applied was about 3 ml at a time, the number of times of application was once a week, and the application was continued every week for about 2 months. As oral care agents, among [1] to [30] above, [1], [2], and [9] to [30] were used as appropriate.

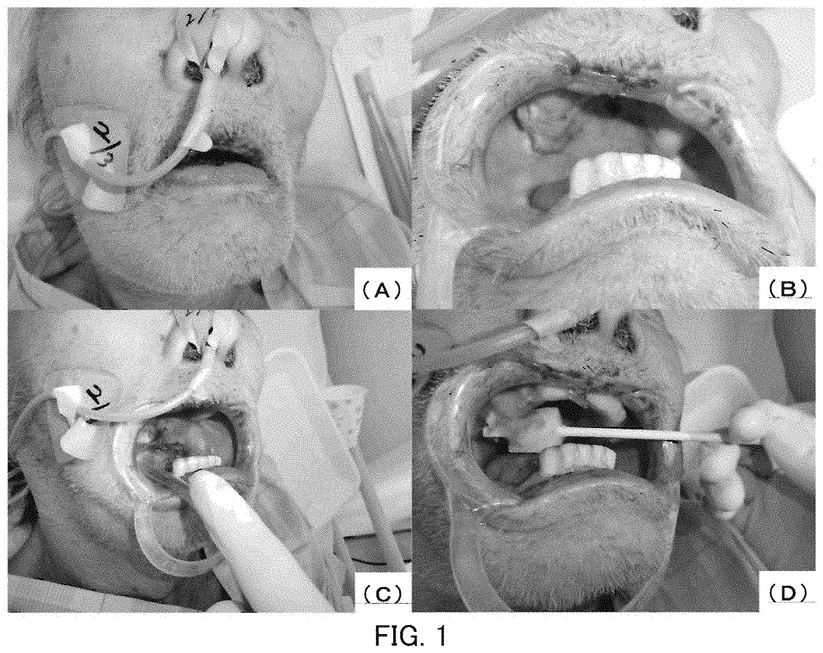

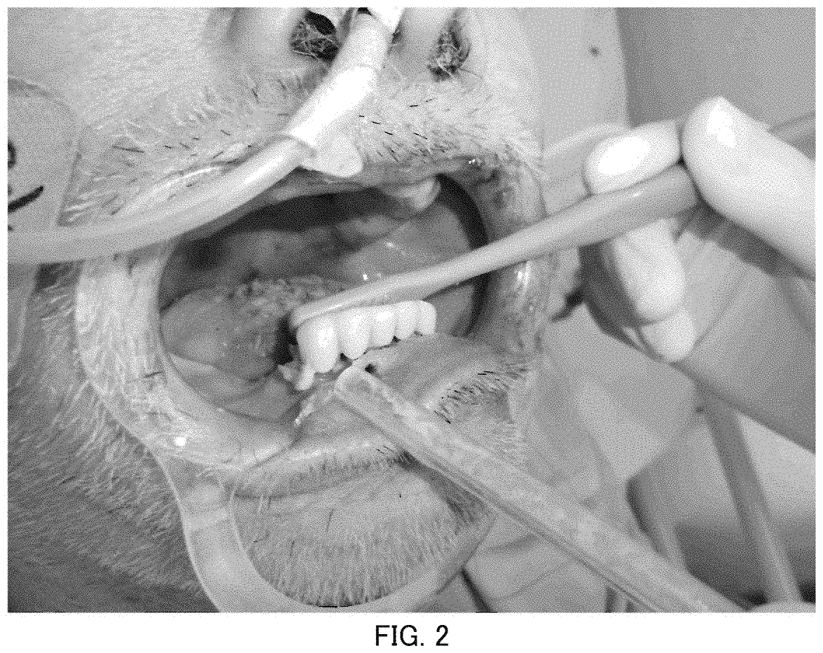

(A) to (D) of FIG. 1 are photographs showing a process of applying an oral care agent to oral deposits mainly composed of sputum and exfoliated epithelium, clot, bacteria, virus, dental plaque, and biofilm adhered to and hardened on the palatal mucosa to soften and removing them with a sponge brush. This procedure can prevent pneumonia and asphyxiation, for example. FIG. 2 is a photograph showing a process of applying an oral care agent to oral deposits mainly composed of sputum and exfoliated epithelium, clot, bacteria, virus, dental plaque, and biofilm adhered to and hardened on the surfaces of teeth and gingivae like calculus to soften and gradually removing by suction while crushing them with a toothbrush. This procedure can treat or prevent caries, periodontal disease, and the like, for example.

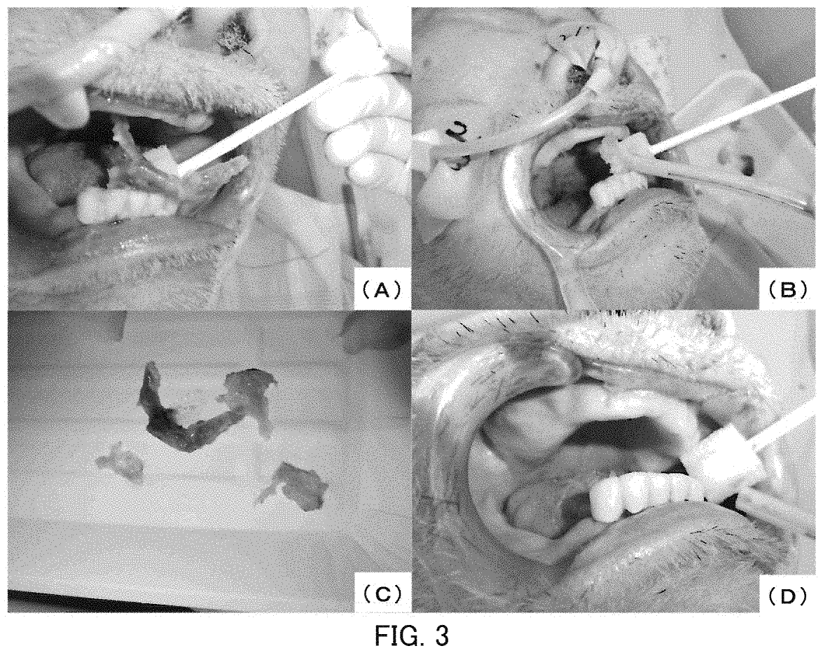

(A) to (D) of FIG. 3 are photographs showing the state after about 5 to 10 minutes from the application of the oral care agent. While oral deposits adhered to and hardened on the surfaces of teeth and gingivae like calculus cause not only caries and periodontal disease but also pneumonia and asphyxiation, the effect of the oral care agent allows the oral deposits to be gradually softened from the peripheral portion and safely removed as a lump without bleeding, aspiration, or asphyxiation by using a sponge brush as shown in (A) to (D) of FIG. 3. In FIG. 3, (C) is a photograph showing oral deposits (sputum and exfoliated epithelium, clot, bacteria, virus, dental plaque, and biofilm) after it was peeled off. In FIG. 3, (D) is a photograph showing the inside of the oral cavity after the oral deposits were peeled off as shown in (C) of FIG. 3. As shown in (C) and (D) of FIG. 3, the oral deposits could be peeled off safely without damaging the oral cavity. Further, before the application of the oral care agent according to the present example, the oral malodor of the patient had an intense odor, but after peeling (removing) the oral deposits as shown in (C) and (D) of FIG. 3, the oral malodor was reduced. This is presumed to be because chlorine dioxide radicals were generated from sodium chlorite included in the oral care agent, and the chlorine dioxide radicals showed the bactericidal deodorizing action.

### REFERENCE EXAMPLE

As shown in Table 1 below, the minimum inhibitory concentration (MIC) and minimum bactericidal concentration (MBC) of MA-T were measured to examine that they act as antimicrobial components. In Table 1 below, the composition of “MA-T107” includes sodium chlorite (radical generator) and benzethonium chloride (radical generating catalyst) by the same mass. The composition of “MA-Tγ” includes sodium chlorite and benzalkonium chloride in a weight ratio of 5:3, as described above. The concentration (ppm) summarized in Table 1 below is the concentration of sodium chlorite, which is the radical source. In Table 1 below, MIC and MBC were measured by the microdilution method.

TABLE 1
List of MICMBC check test for periodontal disease bacteria, etc.

| | | MA-T107 | | MA-Tγ | | |
|---|---|---|---|---|---|---|
| Bacterial species | | MIC | MBC | MIC | MBC | Medium |
| Porphyromonas gingvalis | Periodontal disease | 20 | 20 | 5 | — | BHIHM medium |
| Treponema denticola | Periodontal disease | 25 | 25 | — | — | TYGVS liquid medium |
| Tarnnerella fosythensis | Periodontal disease | 12.5 | 12.5 | — | — | Sheep blood medium supplemented with 0.001% N- |
| | | | | | | acetylmuramic acid |
| A. actinomycetemcomitans | Periodontal disease | 35-45 | 35-50 | 2 | 3 | BHIYE medium/BHI medium |
| Streptococus mutans | Caries | 2.5 | 15 | 3.5 | 3.5 | BHI BHI medium |

### Example 2

As described below, the exfoliated epithelium was immersed in a liquid agent including a radical generator, and it was examined that the radical generator decomposed the exfoliated epithelium.

First, two pieces of approximately the same size were cut with scissors from the exfoliated epithelium taken from the oral cavity of the patient. Each exfoliated epithelium piece was placed in a separate Petri dish. Then, 3 ml of 200 ppm MA-Tγ was added dropwise into one Petri dish and allowed to stand. Note that “200 ppm MA-Tγ” is an aqueous solution containing 200 ppm sodium chlorite (radical generator) and 120 ppm benzalkonium chloride (radical generating catalyst) as described in Example 1. Instead of 3 ml of 200 ppm MA-Tγ, 3 ml of purified water was dropped into the other Petri dish, and the mixture was allowed to stand. Then, the exfoliated epithelial piece in each of the Petri dishes was visually observed over time.

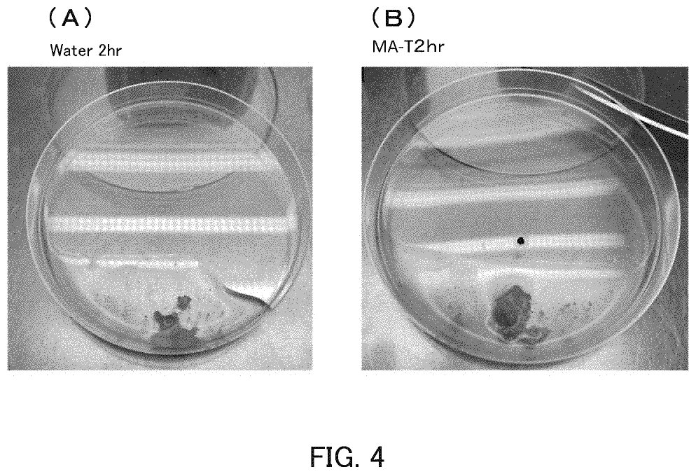

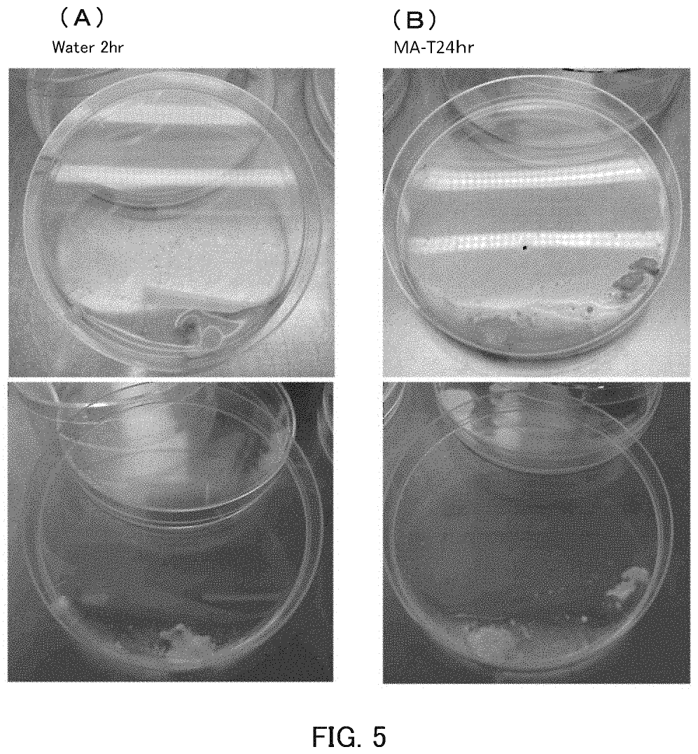

The photographs of FIGS. 4 and 5 show the results of observation of the exfoliated epithelial pieces in Petri dishes. In FIG. 4, (A) is a photograph showing the exfoliated epithelial piece after being immersed in water for 2 hours. In FIG. 4, (B) is a photograph showing the exfoliated epithelial piece after being immersed in 200 ppm MA-Tγ for 2 hours. In FIG. 5, (A) is a photograph showing the exfoliated epithelial piece after being immersed in water for 24 hours. In FIG. 5, (B) is a photograph showing the exfoliated epithelial piece after being immersed in 200 ppm MA-Tγ for 24 hours. As shown in (A) of FIG. 4, the water after 2 hours of immersion of the exfoliated epithelial piece remained almost transparent. On the other hand, as shown in (B) of FIG. 4, 200 ppm MA-Tγ after 2 hours of immersion of the exfoliated epithelial piece was strongly clouded by the appearance of something like fine powders. This difference became more pronounced after 24 hours as shown in (A) and (B) of FIG. 5. As shown in (B) of FIG. 5, the exfoliated epithelial piece immersed in 200 ppm MA-Tγ for 24 hours was decomposed and almost powdered.

In the present example, while the reason why the exfoliated epithelial piece immersed in 200 ppm MA-Tγ was decomposed is not clear, it is considered that, for example, keratin (protein) contained in the exfoliated epithelial piece was hydrolyzed by chlorine dioxide radicals generated from sodium chlorite. As described above, according to the present example, it was verified that the liquid agent including the radical generator decomposes the exfoliated epithelial piece and thus contributes to the removal of the oral deposits.

(Supplementary Notes)

Some or all of the above embodiments and examples may be described as in the following Supplementary Notes, but are not limited thereto.

### (Supplementary Note 1)

A semi-solid oral care agent that is an agent for removing oral deposits.

### (Supplementary Note 2)

The oral care agent according to Supplementary Note 1, comprising:
a radical generator.

### (Supplementary Note 3)

An oral care agent comprising:
a radical generator, wherein
the oral care agent is an agent for removing oral deposits.

### (Supplementary Note 4)

The oral care agent according to Supplementary Note 3, wherein the oral care agent is liquid.

### (Supplementary Note 5)

The oral care agent according to any one of Supplementary Notes 2 to 4, wherein
the radical generator is at least one material selected from the group consisting of oxo acid, oxo acid ion, and oxo acid salt.

### (Supplementary Note 6)

The oral care agent according to Supplementary Note 5, wherein
the oxo acid is a halogen oxo acid.

### (Supplementary Note 7)

The oral care agent according to Supplementary Note 6, wherein
the halogen oxo acid is at least one material selected from the group consisting of chlorine oxo acid, bromine oxo acid, and iodine oxo acid.

### (Supplementary Note 8)

The oral care agent according to Supplementary Note 6 or 7, wherein
the halogen oxo acid is at least one material selected from the group consisting of hypohalous acid, halous acid, halogen acid, and perhalogen acid.

### (Supplementary Note 9)

The oral care agent according to Supplementary Note 6 or 7, wherein
the halogen oxo acid is at least one material selected from the group consisting of hypochlorous acid, chlorous acid, chloric acid, perchloric acid, hypobromous acid, bromous acid, bromic acid, perbromic acid, hypoiodous acid, iodous acid, iodic acid, and periodic acid.

### (Supplementary Note 10)

The oral care agent according to any one of Supplementary Notes 2 to 9, further including:
a radical generating catalyst, wherein
the radical generating catalyst is a substance that catalyzes radical generation from the radical generator comprised in the oral care agent.

### (Supplementary Note 11)

The oral care agent according to Supplementary Note 10, wherein
the radical generating catalyst comprises at least one material selected from the group consisting of ammonium, amino acids, proteins, peptides, phospholipids, and salts thereof.

### (Supplementary Note 12)

The oral care agent according to Supplementary Note 11, wherein
the ammonium is an ammonium salt represented by following chemical formula (XI):

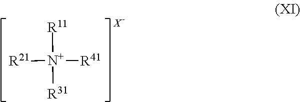

where in the chemical formula (XI),
R¹¹, R²¹, R³¹, and R⁴¹ are each a hydrogen atom or an aromatic ring, or an alkyl group, the alkyl group optionally comprises an ether bond, a carbonyl group, an ester bond, or an amide bond, or an aromatic ring,
R¹¹, R²¹, R³¹, and R⁴¹ are identical to or different from each other,
two or more groups of R¹¹, R²¹, R³¹, and R⁴¹ optionally are integrated to form a ring structure with N⁺ to which the two or more groups are bonded, and the ring structure is saturated or unsaturated and aromatic or non-aromatic, and optionally have one or more substituents, and
X⁻ is an anion.

### (Supplementary Note 13)

The oral care agent according to Supplementary Note 12, wherein
the ammonium salt represented by the chemical formula (XI) is an ammonium salt represented by following chemical formula (XII):

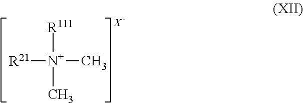

where in the chemical formula (XII),
R¹¹¹ is an alkyl group having 5 to 40 carbon atoms, and optionally comprises an ether bond, ketone (carbonyl group), an ester bond, or an amide bond, a substituent, or an aromatic ring, and
R²¹ and X⁻ are same as the R²¹ and the X⁻ in the chemical formula (XI), respectively.

### (Supplementary Note 14)

The oral care agent according to any one of Supplementary Notes 11 to 13, wherein
the ammonium is at least one material selected from the group consisting of benzethonium chloride, benzalkonium chloride, hexadecyltrimethylammonium chloride, tetramethylammonium chloride, ammonium chloride, methylammonium chloride, tetrabutylammonium chloride, cetylpyridinium chloride, hexadecyltrimethylammonium bromide, dequalinium chloride, edrophonium, didecyldimethylammonium chloride, benzyltriethylammonium chloride, oxytropium, carbachol, glycopyrronium, safranin, sinapine, tetraethylammonium bromide, hexadecyltrimethylammonium bromide, suxamethonium, sphingomyelin, ganglioside GM1, denatonium, trigonelline, neostigmine, paraquat, pyridostigmine, phellodendrine, pralidoxime methiodide, betaine, betanin, bethanechol, betalain, lecithin, adenine, guanine, cytosine, thymine, uracil, and cholines.

### (Supplementary Note 15)

The oral care agent according to Supplementary Note 12, wherein
the ammonium salt represented by the chemical formula (XI) is an ammonium salt represented by following chemical formula (XIV):

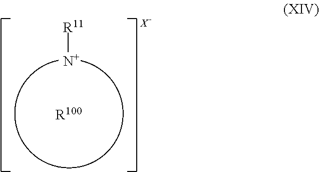

where in the chemical formula (XIV),
R¹⁰⁰ optionally forms a ring structure, which is saturated or unsaturated, aromatic or non-aromatic, and optionally have one or more substituents, and
R¹¹ and X⁻ are the same as the R¹¹ and the X⁻ in the chemical formula (XI), respectively.

### (Supplementary Note 16)

The oral care agent according to any one of Supplementary Notes 10 to 15, wherein
the radical generating catalyst has a Lewis acidity of no lower than 0.4 eV.

### (Supplementary Note 17)

The oral care agent according to any one of Supplementary Notes 1 to 16, wherein
the oral deposit is a substance adhered to oral mucosa, tongue, gingivae, or teeth.

### (Supplementary Note 18)

The oral care agent according to any one of Supplementary Notes 1 to 17, wherein
the oral deposit is at least one substance selected from the group consisting of sputum, crust, exfoliated epithelium, clot, bacteria, virus, dental plaque, and biofilm.

### (Supplementary Note 19)

The oral care agent according to any one of Supplementary Notes 1 to 18, comprising:
at least one of an antimicrobial component or an antiviral component.

### (Supplementary Note 20)

The oral care agent according to any one of Supplementary Notes 1 to 19, wherein the oral care agent is an agent for prevention or treatment of an oral disease.

### (Supplementary Note 21)

The oral care agent according to Supplementary Note 20, wherein
the disease is caries, periodontal disease, stomatitis, or oral candidiasis.

### (Supplementary Note 22)

The oral care agent according to any one of Supplementary Notes 1 to 21, wherein the oral care agent is used for prevention or treatment of infectious disease, suppression of seriousness, or suppression of infection to other people.

### (Supplementary Note 23)

The oral care agent according to Supplementary Note 22, wherein
the infectious disease is a respiratory tract infection.

### (Supplementary Note 24)

Use of the radical generator for producing the oral care agent according to any one of Supplementary Notes 2 to 16.

### (Supplementary Note 25)

Use of the radical generator and the radical generating catalyst for producing the oral care agent according to any one of Supplementary Notes 10 to 16.

### (Supplementary Note 26)

The use according to Supplementary Note 24 or 25, wherein
the oral care agent is the oral care agent according to any one of Supplementary Notes 17 to 23.

### (Supplementary Note 27)

Use of a semi-solid oral care agent for removing oral deposits.

### (Supplementary Note 28)

The use according to Supplementary Note 27, wherein
the semi-solid oral care agent is the oral care agent according to any one of Supplementary Notes 1 to 23.

### (Supplementary Note 29)

A method for removing oral deposits, including:
using a semi-solid oral care agent.

### (Supplementary Note 30)

The method for removing oral deposits according to Supplementary Note 29, wherein
the semi-solid oral care agent is the oral care agent according to any one of Supplementary Notes 1 to 23.

### (Supplementary Note 31)

A method for preventing or treating an oral disease, including:
removing oral deposits using the oral care agent according to any one of Supplementary Notes 1 to 23.

### (Supplementary Note 32)

The method for preventing or treating an oral disease according to claim 31, wherein
the disease is caries, periodontal disease, stomatitis, or oral candidiasis.

### (Supplementary Note 33)

A method for preventing or treating an infectious disease, suppressing seriousness, or suppressing infection to other people, including:
removing oral deposits using the oral care agent according to any one of Supplementary Notes 1 to 23.

### (Supplementary Note 34)

The method for preventing or treating an infectious disease, suppressing seriousness, or suppressing infection to other people according to Supplementary Note 33, wherein
the infectious disease is a respiratory tract infection.

### (Supplementary Note 35)

Use of the oral care agent according to any one of Supplementary Notes 1 to 23 for prevention or treatment of an oral disease, wherein
the oral care agent according to any one of Supplementary Notes 1 to 23 is used to remove oral deposits.

### (Supplementary Note 36)

The use according to Supplementary Note 35, wherein
the disease is caries, periodontal disease, stomatitis, or oral candidiasis.

### (Supplementary Note 37)

Use of the oral care agent according to any one of Supplementary Notes 1 to 23 for prevention or treatment of an infectious disease, suppression of seriousness, or suppression of infection to other people.

### (Supplementary Note 38)

The use according to Supplementary Note 37, wherein
the infectious disease is a respiratory tract infection.

While the present invention has been described above with reference to illustrative embodiments and examples, the present invention is by no means limited thereto. Various changes and variations that may become apparent to those skilled in the art may be made in the configuration and specifics of the present invention without departing from the scope of the present invention.

As described above, according to the present invention, it is possible to provide an oral care agent suitable for removing oral deposits. According to the oral care agent of the present invention, for example, the oral deposits can be removed safely and quickly without damaging the oral surface, and re-adhesion of the oral deposits to the oral mucosa can be suppressed or prevented, thereby facilitating continuous oral care.

This application claims priority from Japanese Patent Application No. 2020-12219 filed on Jul. 16, 2020. The entire subject matter of the Japanese Patent Applications is incorporated herein by reference.

1. A semi-solid oral care agent that is an agent for removing oral deposits.

2. The oral care agent according to claim 1, comprising:
a radical generator.

3. An oral care agent comprising:
a radical generator, wherein
the oral care agent is an agent for removing oral deposits.

4. The oral care agent according to claim 3, wherein the oral care agent is liquid.

5. The oral care agent according to claim 2, wherein
the radical generator is at least one material selected from the group consisting of oxo acid, oxo acid ion, and oxo acid salt.

6. The oral care agent according to claim 5, wherein
the oxo acid is a halogen oxo acid.

7. The oral care agent according to claim 6, wherein
the halogen oxo acid is at least one material selected from the group consisting of chlorine oxo acid, bromine oxo acid, and iodine oxo acid.

8. The oral care agent according to claim 6, wherein
the halogen oxo acid is at least one material selected from the group consisting of hypohalous acid, halous acid, halogen acid, and perhalogen acid.

9. The oral care agent according to claim 6, wherein
the halogen oxo acid is at least one material selected from the group consisting of hypochlorous acid, chlorous acid, chloric acid, perchloric acid, hypobromous acid, bromous acid, bromic acid, perbromic acid, hypoiodous acid, iodous acid, iodic acid, and periodic acid.

10. The oral care agent according to claim 2, further comprising:
a radical generating catalyst, wherein
the radical generating catalyst is a substance that catalyzes radical generation from the radical generator comprised in the oral care agent.

11. The oral care agent according to claim 10, wherein
the radical generating catalyst comprises at least one material selected from the group consisting of ammonium, amino acids, proteins, peptides, phospholipids, and salts thereof.

12. The oral care agent according to claim 11, wherein
the ammonium is an ammonium salt represented by following chemical formula (XI): 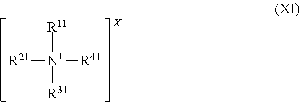
where in the chemical formula (XI),
R¹¹, R²¹, R³¹, and R⁴¹ are each a hydrogen atom or an aromatic ring, or an alkyl group, the alkyl group optionally comprises an ether bond, a carbonyl group, an ester bond, or an amide bond, or an aromatic ring,
R¹¹, R²¹, R³¹, and R⁴¹ are identical to or different from each other,
two or more groups of R¹¹, R²¹, R³¹, and R⁴¹ optionally are integrated to form a ring structure with N⁺ to which the two or more groups are bonded, and the ring structure is saturated or unsaturated and aromatic or non-aromatic, and optionally have one or more substituents, and
X⁻ is an anion.

13. The oral care agent according to claim 12, wherein
the ammonium salt represented by the chemical formula (XI) is an ammonium salt represented by following chemical formula (XII): 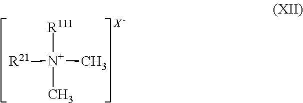
where in the chemical formula (XII),
R¹¹¹ is an alkyl group having 5 to 40 carbon atoms, and optionally comprises an ether bond, ketone (carbonyl group), an ester bond, or an amide bond, a substituent, or an aromatic ring, and
R²¹ and X⁻ are same as the R²¹ and the X⁻ in the chemical formula (XI), respectively.

14. The oral care agent according to claim 11, wherein
the ammonium is at least one material selected from the group consisting of benzethonium chloride, benzalkonium chloride, hexadecyltrimethylammonium chloride, tetramethylammonium chloride, ammonium chloride, methylammonium chloride, tetrabutylammonium chloride, cetylpyridinium chloride, hexadecyltrimethylammonium bromide, dequalinium chloride, edrophonium, didecyldimethylammonium chloride, benzyltriethylammonium chloride, oxytropium, carbachol, glycopyrronium, safranin, sinapine, tetraethylammonium bromide, hexadecyltrimethylammonium bromide, suxamethonium, sphingomyelin, ganglioside GM1, denatonium, trigonelline, neostigmine, paraquat, pyridostigmine, phellodendrine, pralidoxime methiodide, betaine, betanin, bethanechol, betalain, lecithin, adenine, guanine, cytosine, thymine, uracil, and cholines.

15. The oral care agent according to claim 12, wherein
the ammonium salt represented by the chemical formula (XI) is an ammonium salt represented by following chemical formula (XIV): 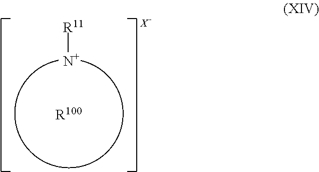
where in the chemical formula (XIV),
R¹⁰⁰ optionally forms a ring structure, which is saturated or unsaturated, aromatic or non-aromatic, and optionally have one or more substituents, and
R¹¹ and X⁻ are the same as the R¹¹ and the X⁻ in the chemical formula (XI), respectively.

16. The oral care agent according to claim 10, wherein
the radical generating catalyst has a Lewis acidity of no lower than 0.4 eV.

17. The oral care agent according to claim 1, wherein
the oral deposit is a substance adhered to oral mucosa, tongue, gingivae, or teeth.

18. The oral care agent according to claim 1, wherein
the oral deposit is at least one substance selected from the group consisting of sputum, crust, exfoliated epithelium, clot, bacteria, virus, dental plaque, and biofilm.

19. The oral care agent according to claim 1, comprising:
at least one of an antimicrobial component or an antiviral component.

20. The oral care agent according to claim 1, wherein the oral care agent is an agent for prevention or treatment of an oral disease.

21. The oral care agent according to claim 20, wherein
the disease is caries, periodontal disease, stomatitis, or oral candidiasis.

22. The oral care agent according to claim 1, wherein the oral care agent is used for prevention or treatment of infectious disease, suppression of seriousness, or suppression of infection to other people.

23. The oral care agent according to claim 22, wherein
the infectious disease is a respiratory tract infection.
